(19) United States
(12) Patent Application Publication
(10) Pub. No.: US 2014/0012304 A1
(43) Pub. Date: Jan. 9, 2014

(54) MULTILAYERED BALLOON

(71) Applicant: MERIT MEDICAL SYSTEMS, INC., South Jordan, UT (US)

(72) Inventors: Fred Lampropoulos, Salt Lake City, UT (US); John William Hall, North Salt Lake, UT (US); Jim Mottola, Salt Lake City, UT (US); Aaron J. Hopkinson, Herriman, UT (US); F. Mark Ferguson, Salt Lake City, UT (US); Rachel Lynn Simmons, Bountiful, UT (US); Richard Brotherton, Park City, UT (US); Wayne L. Mower, Bountiful, UT (US)

(73) Assignee: MERIT MEDICAL SYSTEMS, INC., South Jordan, UT (US)

(21) Appl. No.: 13/829,493

(22) Filed: Mar. 14, 2013

Related U.S. Application Data
(60) Provisional application No. 61/667,795, filed on Jul. 3, 2012.

(51) Int. Cl. A61M 25/10

(52) U.S. Cl. CPC A61M 25/10; A61M 25/1034
USPC 606/192; 29/428

(57) ABSTRACT

A multilayered inflatable medical appliance is disclosed. The appliance may comprise multiple adjacent layers disposed to increase total burst strength, puncture resistance or other properties. One or more layers may be comprised of a rotational spun fiber coating. Further, in some embodiments, additional top coatings may be included. Multilayered constructs may be configured with higher burst strengths and/or puncture resistance as compared to single layer constructs.

### RELATED APPLICATIONS

This application claims priority to U.S. Provisional Application No. 61/667,795 filed on Jul. 3, 2012 and titled “Multilayered Balloon,” which is hereby incorporated by reference in its entirety.

### TECHNICAL FIELD

The current disclosure relates to inflatable medical appliances such as catheter balloons and related components for use in medical procedures such as, but not limited to, angioplasty and valvuloplasty. In some embodiments, an inflatable medical appliance may comprise multiple layers of material. For example, a balloon may be constructed of multiple adjacent layers of material. In other embodiments, one or more layers of a multilayered construct may comprise a matrix of rotational spun fibers. In some instances, multilayered designs may affect the strength of the medical appliance, including burst strength and/or puncture resistance.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a partial cut-away view of a three layer balloon.

FIG. 2 is a front view of a balloon comprising a first balloon layer and an outer film layer.

FIG. 3 is a photograph of a fiber coated balloon.

FIG. 4 is a schematic representation of a first embodiment of a rotational spinning apparatus and a balloon.

FIG. 5 is a schematic representation of a second embodiment of a rotational spinning apparatus and a balloon.

FIG. 6 is a front view of a balloon coated with oriented fibers.

FIG. 7 is a front view of a balloon coated with randomly disposed fibers.

FIG. 8 is a front view of a balloon having a radiopaque band deposited thereon.

FIG. 9 is a partial cut-away view of a balloon coated with fibers, then covered with an outer layer.

FIG. 10 is a perspective view of a balloon coupled to a catheter.

FIG. 11A is a front view of a fiber coated balloon.

FIG. 11B is a front view of a fiber coated unexpanded parison.

FIG. 12A is a scanning electron micrograph (SEM) (at 170× magnification) of a rotational spun nylon balloon coating.

FIG. 12B is an SEM of the nylon coating of FIG. 12A at 950× magnification.

FIG. 13A is an SEM (at 170× magnification) of a rotational spun nylon balloon coating covered with a urethane top coat.

FIG. 13B is an SEM of the nylon coating and urethane top coat of FIG. 13A at 950× magnification.

FIG. 14A is an SEM (at 170× magnification) of a rotational spun matrix of polytetrafluoroethylene (PTFE), polyethylene oxide (PEO), and bismuth bicarbonate.

FIG. 14B is an SEM of the matrix of FIG. 14A at 950× magnification.

### DETAILED DESCRIPTION

Some medical appliances may be configured to be inflated during use or deployment. For example, balloons or balloon catheters may be inflated as part of a minimally invasive therapy. In some embodiments, a balloon may be introduced into a patient's body in a low-profile, deflated configuration, inflated to perform a stage of a therapy, then deflated for removal. Balloons, balloon catheters, or other inflatable medical appliances may be used in connection with angioplasty, valvuloplasty, stent placement or expansion, and so forth. In some instances, balloons may comprise a multilayered design, including embodiments wherein one or more layers comprise a matrix of spun fibers. Multilayered designs may be configured to strengthen or otherwise affect certain properties of the balloon, including mechanical properties such as burst strength and puncture resistance.

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the disclosure, but is merely representative of various embodiments. While the various aspects of the embodiments are presented in drawings, the drawings are not necessarily drawn to scale unless specifically indicated.

The term “balloon” is used broadly throughout this disclosure to refer to a variety of inflatable medical appliances having a variety of shapes, characteristics, and uses. Further, disclosure or concepts provided in connection with embodiments or examples reciting particular shapes, structures, or uses may be analogously applied to any inflatable medical device.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “proximal” and “distal” are used herein to refer to opposite locations on a balloon or another medical device. The proximal end of a medical device is defined as the end closest to the practitioner when the practitioner is manipulating or operating the device. The distal end is the end opposite the proximal end, along the longitudinal direction of the appliance, or the end furthest from the practitioner during use.

A balloon may comprise a wall defining the interior portion of the balloon and separating the interior portion from the external environment. As the balloon is inflated, fluid may be introduced into the interior portion, exerting pressure on the wall of the balloon. In some therapies the wall may be used to exert pressure on structures or objects outside the balloon. For example, a balloon may be inflated within a body lumen at the location of a blockage or other stricture, the wall of the balloon being used to break up the blockage or force it toward the lumen wall. Similarly a balloon may be used to exert an expansive force on a stent, to deploy the stent and force it out to contact with a body lumen wall. In some embodiments balloons may be configured with a “flow through” type design, which may allow blood or other fluids to pass through the balloon while the balloon is inflated. For example, a balloon may be shaped like a hollow cylinder, allowing the balloon to be inflated within a blood vessel to exert pressure on the vessel wall, while still allowing blood to pass through the center of the balloon.

Balloons may be configured for use in connection with high inflation pressures for certain therapies. Inflation pressure, as used herein, refers to the pressure within the interior portion of the balloon. Certain procedures may necessitate relatively high inflation pressure. For example, in some instances valvuloplasty may be performed at relatively high inflation pressures for the relative size of the balloons used in such therapies. For example, it may be desirable to form valvuloplasty balloons ranging from about 26 mm to about 30 mm in diameter such that those balloons can be used with inflation pressures from about 15 ATM to about 17 ATM. Thus, in some embodiments, balloons may be configured to withstand high inflation pressures without bursting or undesirably deforming.

In some embodiments, the exterior wall of a balloon may comprise multiple layers. Constructing a balloon wall of multiple layers may increase the burst strength, puncture resistance, or other properties of the balloon. The type of materials used, the position and thickness of the layers, the size of the balloon, and other factors may impact the overall properties of the balloon.

Balloons may be formed of a variety of materials, including elastomers, polymers, flexible materials, and so forth. Specifically, in some embodiments balloons may be formed of PEBAX, nylon, silicone, or any thermoplastic material. Multilayered balloons may be comprised of multiple layers of the same material, or layers of different materials.

The wall of a multilayered balloon may be comprised of adjacent layers of material. In some instances a balloon wall may be formed of one, two, three, four, or five layers of material. In still other embodiments, a balloon wall may be formed of up to 10 layers of material or more.

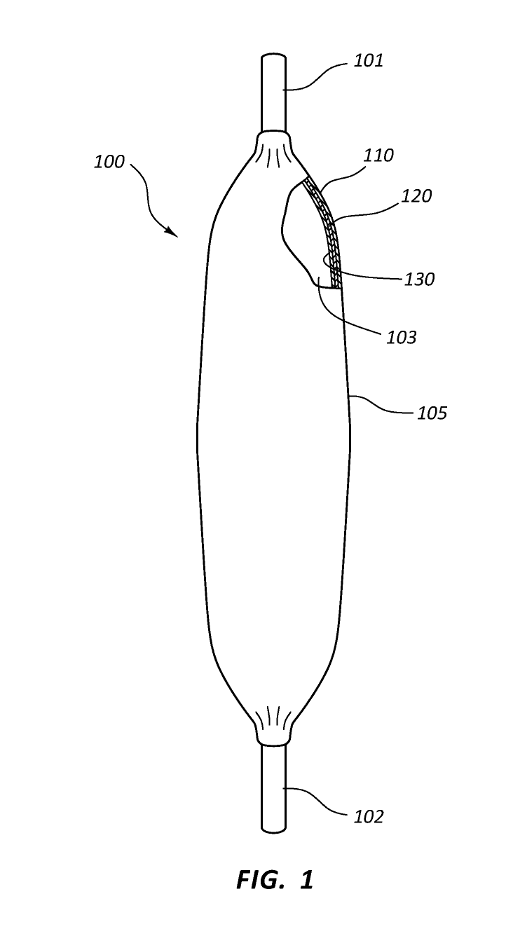

FIG. 1 is a partial cut-away view of a balloon 100 comprising three layers 110, 120, 130. In the illustrated embodiment, the balloon 100 comprises a first leg 101 and a second leg 102. The first 101 and second 102 legs of the balloon 100 may define proximal or distal legs, depending on the orientation of the balloon 100 with respect to the inflation device or other component. In some embodiments an inflation catheter is coupled to one of the first 101 and second 102 legs. Further, one of the first 101 and second 102 legs may be sealed, for example at an end not connected to an inflation catheter. The balloon 100 may further define an interior portion 103. Fluid pressure within the interior portion 103 may tend to inflate the balloon 100.

A balloon wall 105 may define the boundary between the interior portion 103 and the exterior of the balloon 100. In some embodiments, inflation of the balloon 100 will radially expand the balloon wall 105 such that the balloon wall 105 encompasses a larger volume when the balloon 100 is inflated. In the illustrated embodiment, the balloon wall 105 is comprised of three layers: a first layer 110, a second layer 120, and a third layer 130.

Multilayered balloon walls, such as balloon wall 105, may comprise various materials. For example, each of the first layer 110, the second layer 120, and the third layer 130 may be comprised of different materials. In other embodiments, one or more of the layers 110, 120, 130 comprise the same material. Further, balloon walls comprised of greater or fewer than three layers may comprise all layers of the same material or various layers of various materials, including embodiments wherein some layers are the same as others, but not the same as all others.

Multilayered balloon walls, such as balloon wall 105, may exhibit increased strength or other material properties as compared to a single layer wall which is formed of the same material as the multilayered wall. For instance, Example 1, below, compares the burst strength of a balloon having a wall formed of a single PEBAX layer and a balloon having a wall formed of two adjacent layers of PEBAX. Further, additional layers may further increase the burst strength of a balloon. Example 2, below, measures the burst strength of an exemplary balloon having walls formed of three adjacent layers of PEBAX.

Further, a multilayered balloon wall formed of multiple layers of the same material may have different properties than a balloon wall formed of a single layer of the material with the same total thickness. For instance, the multilayered design may have a higher burst strength than the single wall design. A multilayered design may also be more flexible than a single layered design.

In some instances, adjacent layers within a balloon wall may be unconstrained such that they are allowed to slide with respect to each other. For example, in balloon wall 105 one or more of the first layer 110, the second layer 120, and the third layer 130 may be uncoupled to one or more adjacent layers along the length of the balloon 100. In some embodiments the layers 110, 120, 130 may be coupled to each other at discrete points, such as adjacent one or both legs 101, 102 but allowed to slide or move with respect to each other along other portions. For example, the layers 110, 120, 130 may be welded or bonded with an adhesive at one or both legs 101, 102. Coupling one or more entire layers of a multilayered balloon, for example through the use of adhesive, is also within the scope of this disclosure.

A balloon wall 105 comprised of layers 110, 120, 130 which are allowed to move with respect to each other may increase the overall flexibility of the balloon 100 as well as impact mechanical properties, such as burst strength or puncture resistance.

Use of multilayered balloon constructs may facilitate various aspects of therapies involving balloons. For example, as discussed above, a multilayered design may be thinner and/or more flexible than a single layer balloon having comparable burst strength. This may enable the balloon to be folded or otherwise packed into a smaller delivery configuration. Balloons with smaller delivery profiles may be introduced at more locations on the human body than larger balloons, which may facilitate treatment and access. Additionally, smaller profiles may require smaller access openings, which may decrease bleeding, trauma, and complications.

Further, relatively large balloons generally have lower burst strengths than smaller balloons. Use of multilayered constructs may facilitate production and use of larger balloons which are small enough to be introduced at various locations on the body and yet have sufficient burst strengths to facilitate treatment. Moreover, multilayered designs may increase the maximum possible balloon burst strength—while still making the balloon deliverable through the body—increasing the types of therapies which may be performed.

Multilayered balloon constructs may further increase the strength and usability of balloons by decreasing the risk that manufacturing or material defects within the balloon will compromise the integrity of the balloon. In other words, a single layer balloon having a defect in the wall of the balloon will likely have a weak point at the defect. However, it is unlikely that a defect in one layer of a multilayered design will be aligned with a defect in an adjacent layer. Thus, the effect of any single defect may be minimized, as the defect area will be reinforced by portions of adjacent layers which are likely defect free.

Similarly, the outside layer of a multilayer balloon may contact bodily structures or other medical appliances during delivery and use. Such contact may stretch, scratch, pierce, or otherwise weaken the layer. As with material defects, however, these points may be reinforced by adjacent layers which are not compromised by such contact. Conversely, such points on a single layer design may more significantly affect the overall strength of the balloon. Thus, as opposed to a single layer design, a multilayered design may be more robust, particularly for use in potentially damaging conditions.

Multiple layer balloons may be formed by simply inflating balloons within each other. For example, a first balloon may be inflated and a second, deflated balloon inserted into the first balloon. The second balloon may be folded or otherwise disposed in a low-profile delivery configuration prior to insertion into the first balloon. The second balloon may then be inflated within the first balloon to form a balloon comprising a two layered construct. This process may be repeated to form additional layers. Referring specifically to the balloon 100 of FIG. 1, a first balloon formed by the first layer 110 may first be inflated. A second balloon formed by the second layer 120 may be inserted into the first balloon and inflated. A third balloon formed of the third layer 130 may then be inserted into the second balloon and inflated. This procedure creates a three layer balloon 100 wherein none of the three layers 110, 120, 130 are fixedly coupled with respect to each other. Once a multilayered balloon, such as balloon 100, is formed, the entire construct may be deflated and disposed in a low-profile configuration for delivery and use.

Thus, multiple layer balloons may be formed by radially folding, wrapping, or compressing a first balloon and inserting it inside a second balloon and inflating the first balloon into the second balloon. Again, this process may be repeated by inserting another folded, wrapped, or compressed balloon into the previous balloons. This process may allow each layer of the eventual balloon construct to be formed independently, allowing for the properties of each layer to be individually optimized or varied. For example, in some instances, a multilayered balloon, such as balloon 100, may include an outer layer, such as layer 110, with increased lubricity or abrasion resistance. Inner layers, such as 120 and/or 130, may be configured to provide strength in various directions (i.e., axial strength, hoop strength, and so forth), flexibility, resistance to creep, and other properties to the construct.

In some processes, to aid in inserting a folded, wrapped, or compressed balloon into the other balloon or balloons of a construct, the outer balloon or balloons may be trimmed at the bottom of the balloon near the leg, to widen the opening available to insert inner layers of balloons. Alternatively, balloons may be blown or otherwise formed with different leg diameters to allow balloon legs to fit inside each other for each layer.

During formation of a multilayered construct, air may be removed between layers by inflating the innermost balloon to force any trapped air out from between the layers and to close any gaps or voids between layers. While the innermost balloon is so inflated, the legs of the balloons may be sealed with respect to each other. Once the balloon is deflated, the layers will be sealed such that air or other fluids cannot create gaps between adjacent layers. The legs may be sealed, for example, by welding each layer together at or adjacent the legs or bonding them together with adhesive at or adjacent the legs. Additionally, vacuum pressure may be used on the outside of the balloons to draw out air not forced out during inflation of the interior balloon and to keep air from migrating into the construct during the sealing process.

Additionally, balloons may be formed within each other in the first instance. For example, a first balloon may be formed by heating and inflating a first segment of material, such as a parison, including embodiments wherein the parison is expanded within a mold. A second parison, or segment of unexpanded material, could then be inserted into the first balloon, heated and expanded in a similar manner. As above, this process may be repeated to form additional layers.

It is within the scope of this disclosure to perform all or any sub combination of steps for forming multilayered balloon constructs within a vacuum or partial vacuum, in order to reduce the occurrence of air or other materials becoming trapped between adjacent layers. Additionally or alternatively, a vacuum or partial vacuum may be applied after construction of the multilayered balloon.

As described above, multilayered balloon constructs may be formed of multiple layers of the same material, or multiple layers of different materials. In some instances, layers of a single construct may be formed of materials with similar elasticity. In such instances, there may be an additive effect—with respect to properties such as burst strength—when adding layers of materials with similar elasticity. In other words, constructs with more layers (wherein each layer has a similar elasticity) may be stronger than constructs of a single layer. As suggested by comparing the results of one, two, and three layer designs in Examples 1 and 2, each subsequent layer may not add as much strength to the overall construct as the first additional layer.

In some embodiments, the modulus of elasticity of one or more layers of a multilayered construct may be within a particular range with respect to the modulus of elasticity of another layer of the construct. In one embodiment, the modulus of elasticity of any of the layers of a construct differs by no more than 20%. In other embodiments, the modulus of elasticity differs by no more than 10%. In yet other embodiments, the modulus of elasticity differs by no more than 5%.

In contrast to the additive effect seen when multiple layers have similar elasticity, constructs having multiple layers with different elasticity may not exhibit an additive effect. In other words, a multilayered construct having a first layer which is much less flexible than other layers may be controlled only by the least flexible layer, with additional layers having little impact on properties such as the burst strength of the balloon. Example 3, below, illustrates this effect with a PEBAX balloon surrounded by a stainless steel mesh.

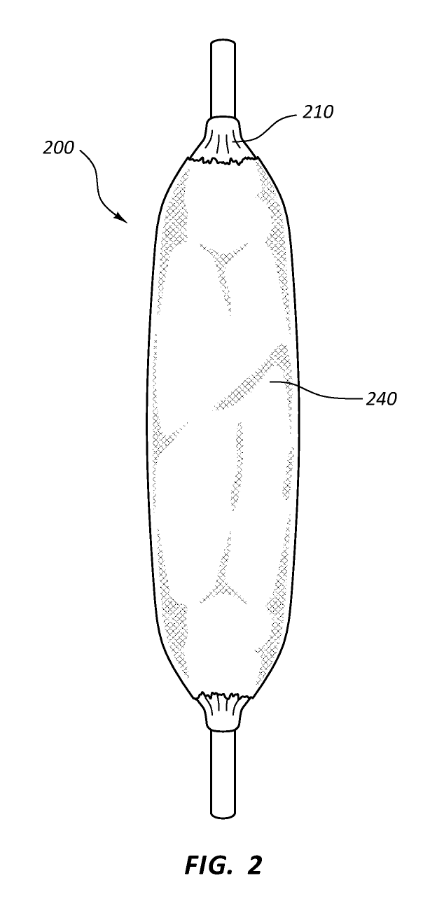

In addition to the methods described above, multilayered designs may also be constructed by coating (such as by dipping or spraying) or wrapping an initial layer with additional material. FIG. 2 is a front view of a balloon 200 comprising a first balloon layer 210 and an outer film layer 240. The balloon 200 of FIG. 2 may resemble components of the balloon 100 of FIG. 1 in some respects. It will be appreciated that all the illustrated embodiments have analogous features. Accordingly, like features are designated with like reference numerals, with the leading digits incremented to “2.” (For instance, the balloon is designated “100” in FIG. 1 and an analogous balloon is designated as “200” in FIG. 2.) Relevant disclosure set forth above regarding similarly identified features thus may not be repeated hereafter. Moreover, specific features of the balloon and related components shown in FIG. 2 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the balloon and related components of FIG. 2. Any suitable combination of the features, and variations of the same, described with respect to the balloon and components illustrated in FIG. 1, can be employed with the balloon and components of FIG. 2, and vice versa. This pattern of disclosure applies equally to further embodiments depicted in subsequent figures and described hereafter.

In the embodiment of FIG. 2, the balloon 200 comprises a first balloon layer 210 covered with a film layer 240. The balloon 200 may comprise any material disclosed herein and the film layer 240 may comprise a wrapped film layer, a dipped film layer, a sprayed film layer, and so forth. For example, the first balloon layer 210 may comprise PEBAX while the film layer comprises a Kapton film disposed thereon.

Layers, such as film layer 240, disposed on a first balloon layer, such as first balloon layer 210, may be coupled to the initial layer at the ends of the balloon, along the length of the balloon, or both. As with the layers described above, coated or wrapped layers may be configured to increase particular properties of the construct. In some instances, such layers may be configured to provide a protective covering to the balloon and/or increase the puncture resistance of the balloon.

As recited throughout, multilayered designs may be configured to impact the mechanical properties of a balloon construct. In some embodiments, layers may impact multiple properties. For example, a two layer design may both (1) increase the burst strength, as suggested in Example 1 and the disclosure above and (2) provide a protective outer coating which may increase the puncture resistance of the balloon. It will be appreciated by one of skill in the art having the benefit of this disclosure that many of the methods and constructs described above to increase burst strength may likewise increase puncture resistance or other mechanical properties.

As further detailed below, a balloon may be coated or covered with serially deposited micro-fibers and/or nano-fibers. In some embodiments these fibers are deposited directly on the balloon. It is also within the scope of the disclosure to obtain a mat of serially deposited fibers which are subsequently applied to a balloon.

Serially deposited fiber mats or lattices, whether or not deposited directly on a balloon, refer to structures composed at least partially of fibers successively deposited on a collector, on a substrate, on a base material, and/or on previously deposited fibers. In some instances the fibers may be randomly disposed, while in other embodiments the alignment or orientation of the fibers may be somewhat controlled or may follow a general trend or pattern. Regardless of any pattern or degree of fiber alignment, because the fibers are deposited on the collector, substrate, base material, and/or previously deposited fibers, the fibers are not woven, but rather serially deposited. Because such fibers are configured to create a variety of structures, as used herein, the terms “mat” and “lattice” are intended to be broadly construed as referring to any such structure, including tubes, spheres, sheets, coatings deposited directly on a balloon or other medical device, and so on. Furthermore, the term “membrane” as used herein refers to any structure comprising serially deposited fibers having a thickness which is smaller than at least one other dimension of the membrane. Examples of membranes include, but are not limited to, serially deposited fiber mats or lattices forming sheets, strips, tubes, spheres, covers, layers, and so forth.

Rotational spinning is one example of how a material may be serially deposited as fibers. One embodiment of a rotational spinning process comprises loading a polymer solution or dispersion into a cup or spinneret configured with orifices on the outside circumference of the spinneret. The spinneret is then rotated, causing (through a combination of centrifugal and hydrostatic forces, for example) the flowable material within the spinneret to be expelled from the orifices. The material may then form a “jet” or “stream” extending from the orifice, with drag forces tending to cause the stream of material to elongate into a small diameter fiber. The fibers may then be deposited on a collection apparatus, a substrate, or other fibers. Once collected, the fibers may be dried, cooled, sintered, or otherwise processed to set the structure or otherwise harden the fiber mat. For example, polymeric fibers rotational spun from a dispersion may be sintered to remove solvents, fiberizing agents, or other materials as well as to set the structure of the mat. In one embodiment, for instance, an aqueous polytetrafluoroethylene (PTFE) dispersion may be mixed with polyethylene oxide (PEO) (as a fiberizing agent) and water (as a solvent for the PEO), and the mixture rotational spun. Sintering by heating the collected fibers may set the PTFE structure and evaporate off the water and PEO. Exemplary methods and systems for rotational spinning can be found in U.S. patent application Ser. No. 13/742,025, filed on Jan. 15, 2013, and titled “Rotational Spun Material Covered Medical Appliances and Methods of Manufacture,” which is herein incorporated by reference in its entirety.

Electrospinning is another embodiment of how a material may be serially deposited as fibers. One embodiment of an electrospinning process comprises loading a polymer solution or dispersion into a syringe coupled to a syringe pump. The material is forced out of the syringe by the pump in the presence of an electric field. The material forced from the syringe may elongate into fibers that are then deposited on a grounded collection apparatus, such as a collector or substrate. The system may be configured such that the material forced from the syringe is electrostatically charged, and thus attracted to the grounded collection apparatus. As with rotational spinning, once collected, the fibers may be dried, cooled, sintered, or otherwise processed to set the structure or otherwise harden the fiber mat. For example, polymeric fibers electrospun from a dispersion may be sintered to remove solvents, fiberizing agents, or other materials as well as to set the structure of the mat. As in rotational spinning, one embodiment of electrospinning comprises electrospinning an aqueous PTFE dispersion mixed with PEO and water (as a solvent for the PEO). Sintering by heating the collected fibers may set the PTFE structure and evaporate off the water and PEO. Exemplary methods and systems for electrospinning medical devices can be found in U.S. Provisional Patent Application No. 61/703,037, filed on Sep. 19, 2012, and titled “Electrospun Material Covered Medical Appliances and Methods of Manufacture,” and U.S. patent application Ser. No. 13/360,444, filed on Jan. 27, 2012, and titled “Electrospun PTFE Coated Stent and Method of Use,” both of which are hereby incorporated by reference in their entireties.

Rotational spinning and/or electrospinning may be utilized to create a variety of materials or structures comprising serially deposited fibers. The microstructure or nanostructure of such materials, as well as the porosity, permeability, material composition, rigidity, fiber alignment, and so forth, may be controlled or configured to promote biocompatibility or influence interactions between the material and cells or other biologic material. A variety of materials may be serially deposited through processes such as rotational spinning and electrospinning, for example, polymers, ceramics, metals, materials which may be melt-processed, or any other material having a soft or liquid form. A variety of materials may be serially deposited through rotational spinning or electrospinning while the material is in a solution, dispersion, molten or semi-molten form, and so forth. The present disclosure may be applicable to any material discussed herein being serially deposited as fibers onto any substrate or in any geometry discussed herein. Thus, examples of particular materials or structures given herein may analogously be applied to other materials and/or structures.

Rotational spinning, electrospinning, or other analogous processes may be used to create serially deposited fiber mats as disclosed herein. Throughout this disclosure, examples may be given of serially deposited fiber mats generally, or the examples may specify the process (such as rotational spinning or electrospinning) utilized to create the serially deposited fiber mat. It is within the scope of this disclosure to analogously apply any process for creating serially deposited fibers to any disclosure or example below, regardless of whether the disclosure specifically indicates a particular mat was formed according to a particular process.

Serially deposited coatings, including rotational spun or electrospun coatings may be applied to any balloon substrate and may be configured to provide additional strength to the balloon, increase the puncture resistance of the balloon, provide a lubricious coating, and so forth. Serially depositing fibers may be used to coat a balloon with a matrix of fibers, including nano-fibers and/or micro-fibers. The fibers may be deposited directly on the exterior surface of the balloon to be coated.

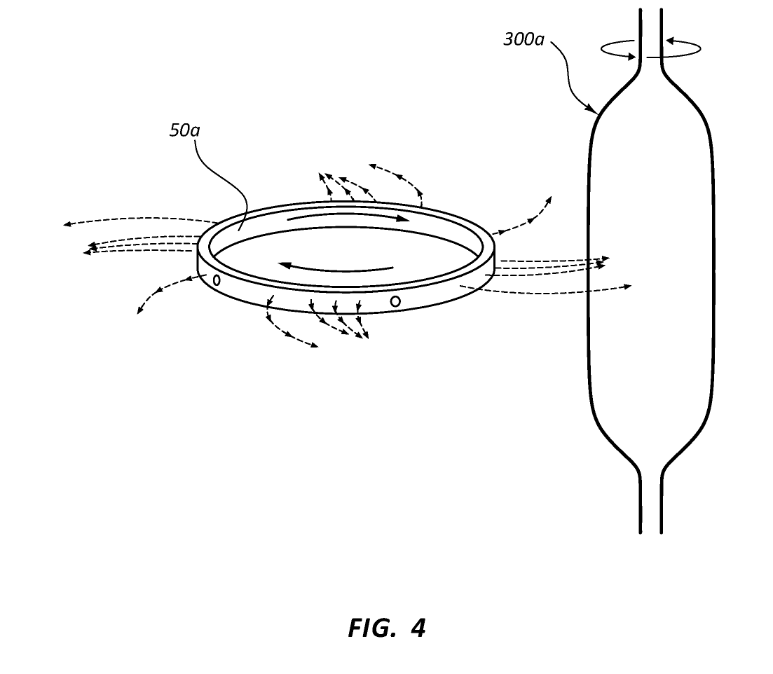

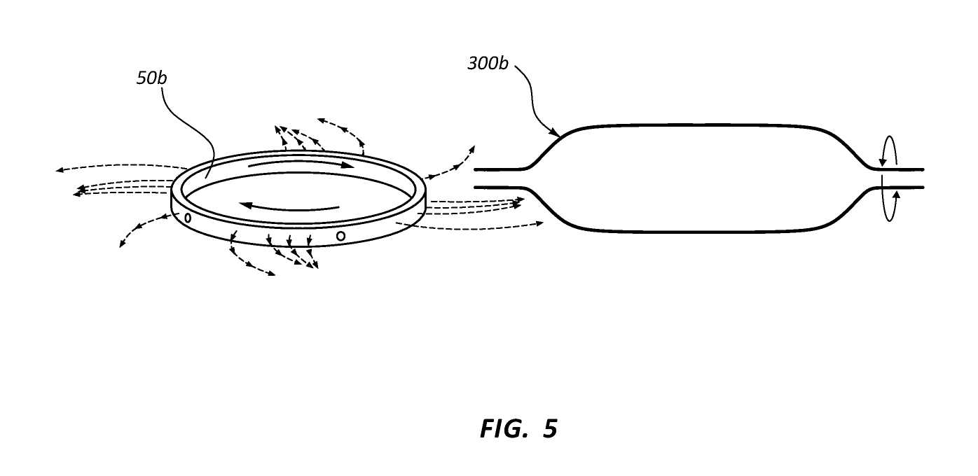

Specific examples and disclosure below relate to rotational spinning fibers on a balloon substrate. FIGS. 4 and 5, for example, are schematic representations of embodiments of rotational spinning apparatuses and balloons. Notwithstanding the specific examples herein, analogous use of other methods of serially depositing fibers, such as electrospinning, are also within the scope of this disclosure. Further, disclosure provided in connection with coating balloons with serially deposited fibers is relevant to any disclosure above regarding multilayered balloons. In some embodiments a multilayered balloon may be coated with serially deposited fibers. In some embodiments, each layer of a multilayered balloon may each be coated with serially deposited fibers before the layers are assembled into a single construct.

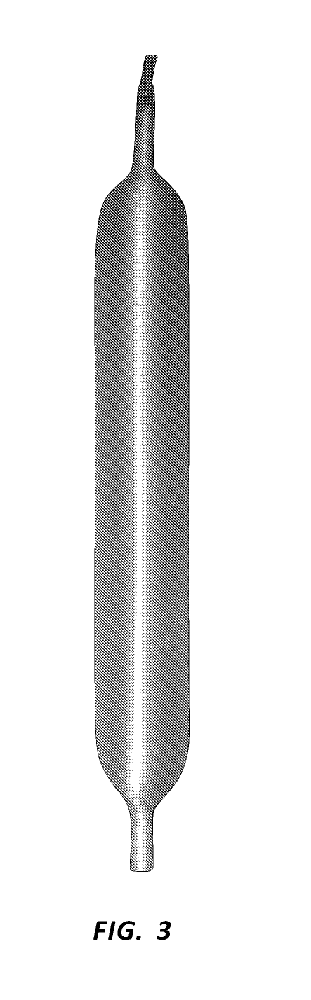

Rotational spun fibers may have relatively small diameters and masses which may allow the fibers to evenly coat the contours of balloon. FIG. 3 is a photograph of a PEBAX balloon coated with rotational spun fibers. As can be seen in FIG. 3, the rotational spun fibers may be deposited such that they evenly coat the entire surface of the balloon, including the contours of the portions of the balloon which transition between a large diameter and a small diameter. Thus, in some embodiments, rotational spun coatings may be applied at a relatively uniform thickness and at a relatively uniform fiber density over the surface of the balloon.

In some embodiments, a balloon may be coated with rotational spun fibers according to a procedure comprised as follows. First, an inflated balloon may be placed within a rotational spinning apparatus, or in proximity to a rotational spinning spinneret. Material to be rotational spun may then be loaded into the spinneret reservoir. The spinneret may then be spun and the balloon coated with fibers. The thickness of the coating may be controlled by the amount of time the spinneret is allowed to run. The balloon may then be removed. The spun material may be cured (for example to remove solvent) if necessary. In some instances, curing may comprise heating the construct, including heating the construct such that the coating is sintered. Certain materials, such as nylon, for example, may not require sintering. Additionally, in some embodiments the balloon may first be dipped or spray coated with an additional layer of material configured to bond the fibers to the substrate.

Furthermore, the deposition of fibers on the balloon may also be controlled by rotating the balloon during the coating procedure. This may facilitate uniform and even coating of the balloon. FIG. 4 is a schematic representation of a first embodiment of a rotational spinning apparatus (comprising a spinneret 50a) and a balloon 300a, wherein the balloon 300a is configured to rotate about an axis which is substantially parallel to the axis of rotation of the spinneret 50a. The dotted lines extending from the spinneret 50a show potential directions of fibers leaving the spinneret 50a and being deposited on the balloon 300a. (The fiber paths are exemplary only. In some embodiments fibers may loop all the way around the spinneret 50a before being deposited.)

Additionally, FIG. 5 is a schematic representation of a second embodiment of a rotational spinning apparatus (comprising a spinneret 50b) and a balloon 300b, wherein the balloon 300b is configured to rotate about an axis which is substantially orthogonal to the axis of rotation of the spinneret 50b.

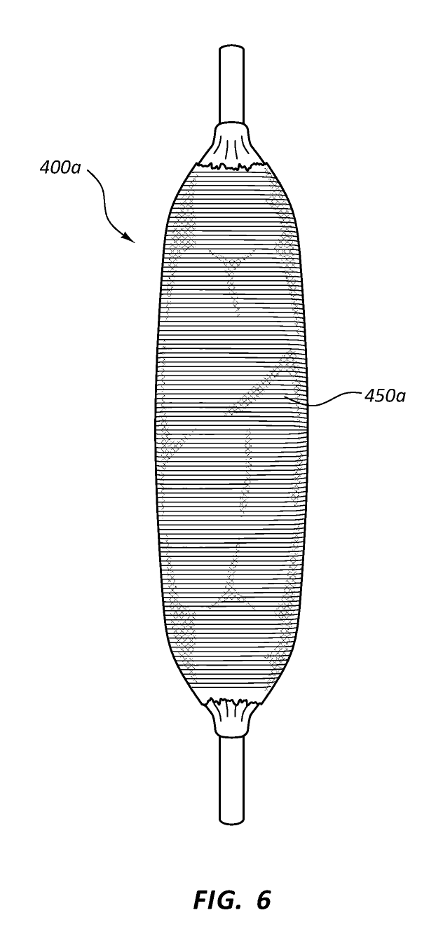

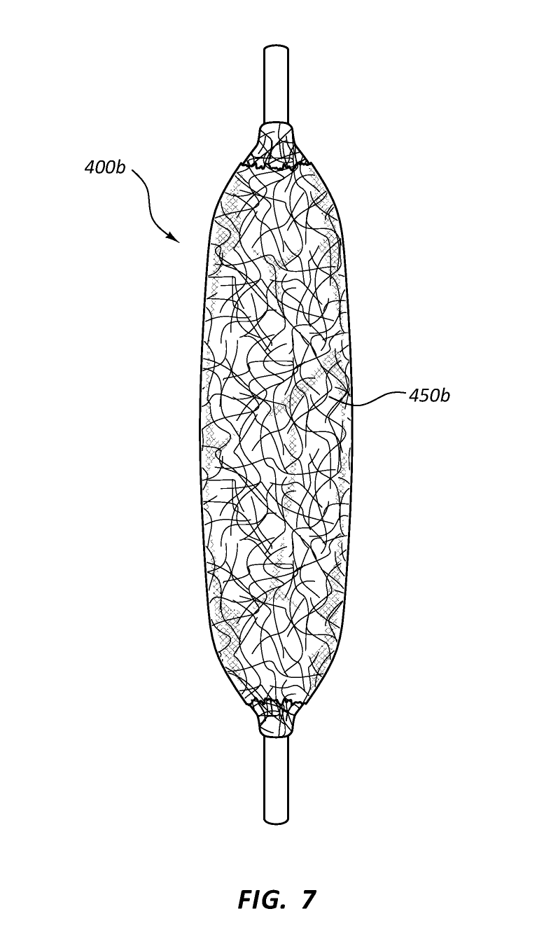

During rotational spinning or otherwise serially depositing fibers, controlling the direction and/or speed of rotation of the balloon may affect the deposition of fibers on the balloon. For example, in embodiments wherein the balloon is configured to rotate about an axis parallel to the axis of rotation of the spinneret (as in FIG. 4), the fibers may be deposited in an oriented arrangement, with the fibers tending to wrap around a circumference of the balloon. By comparison, the apparatus of FIG. 5 may result in a more random arrangement of fibers on the balloon. For example, FIG. 6 is a front view of a balloon 400a coated with oriented fibers 450a and FIG. 7 is a front view of a balloon 400b coated with randomly disposed fibers 450b.

Varying the rotational speed of the balloon during the coating process may affect the properties of the resultant coating. In some embodiments the balloon may be rotated at between about 100 RPM and about 10,000 RPM or more, including from about 200 RPM to about 5000 RPM, or from about 1000 RPM to about 3000 RPM. In some embodiments, higher rotational speeds may result in a relatively more aligned fiber pattern on the balloon.

In some embodiments fibers may be deposited in multiple layers, varying the orientation and/or other characteristics between layers. The type, size, deposition pattern, and other aspects of the fibers may be configured to affect the strength, elasticity, puncture resistance, and other properties of the coated balloon. When spinning polymeric materials from a solution, in some embodiments the higher the concentration of polymer in the solution, the stronger the resultant fibers may be.

Generally, a rotational spun (or other serially deposited) coating on a balloon can vary in thickness and density depending on the desired characteristics of the coat and the intended application. In some embodiments, a coating of rotational spun fibers may be from about 25 microns to about 800 microns thick, including from about 200 microns to about 500 microns. The diameter of deposited fibers may likewise vary depending on the desired characteristics of the coating. In some embodiments the fibers may be on the nanoscale, meaning smaller than one micron in diameter. In other embodiments the fibers may be on the microscale, meaning smaller than one millimeter in diameter. In certain embodiments the fibers may be from about 500 nanometers to about 1.5 microns in diameter.

Additionally, when coating balloons, a wide variety of input values may be used and manipulated to control the properties of the rotational spun coat. In some embodiments the spinneret may be rotated from about 2500 RPM to about 8000 RPM or more, depending on the characteristics of the material to be spun. Further, a variety of orifice sizes are within the scope of this disclosure. In some embodiments, the orifices may comprise needles from about 20 gauge to about 32 gauge in size. In two exemplary procedures, a spinneret configured with 20 gauge needle orifices was spun at about 2700 RPM and a spinneret configured with 26 gauge needle orifices was spun at about 7500 RPM while coating balloons.

A variety of materials may be rotational spun to coat balloons. In some instances the material may be dissolved in a solvent prior to spinning while in other embodiments the material may be heated until it is melted (or otherwise flowable) and then spun. For example, nylon 6 and nylon 6-6 may be solvent spun. Polyethylene and polypropylene may be either melt spun or solvent spun. PTFE and Kevlar may be solvent spun. These materials are exemplary only; a wide variety of materials, including polymers and other materials may be rotational spun, either as dissolved in a solution or dispersion or by melting or partially melting the material. Additionally, carbon fibers may be rotational spun from solution form in some embodiments.

Various polymers may be dissolved in various solvents to create solutions to be rotational spun. For example, the rotational spun nanofibers or microfibers may comprise a material selected from at least one of the following: polyamide, aromatic polyimide, polyethylene and polypropylene. In one embodiment, a polyamide, such as nylon 6, may be dissolved in hexafluoro propanol (such as 1,1,1,3,3,3-hexafluoro-2-propanol) to form a solution. Nylon 6 may similarly be dissolved in formic acid to create a solution to be rotational spun. Such solutions (of either solvent) may comprise from about 5% to about 30% polyamide by weight, including from about 10% to about 20% polyamide by weight or from about 15% to about 25% polyamide by weight. Further exemplary concentrations of these two examples are recited in connection with Examples 4 and 5, below. Nylon 6 or other polymers may additionally be dissolved in phenol, methanol, or hydrochloric acid, though creation of such solutions may include additional steps of heating or agitation to get the polymer into solution. Additionally, polyethylene, including ultra high molecular weight polyethylene, may be dissolved in decahydronaphthalene or xylene. Aramid powder (aromatic polyimide), or other aromatic polymers, may be dissolved in hexafluoro propanol, hydrochloric acid, acetic acid, or sulfuric acid. In the case of sulfuric acid as a solvent, aramid powder may dissolve at room temperature to create a very viscous solution at a low weight percent of aramid powder.

In some embodiments, a band or portion of the balloon may be coated with a layer of radiopaque material. For example, a balloon such as a PEBAX balloon may have a coating or partial coating of a material such as bismuth deposited thereon. A band of bismuth may be deposited by rotational spinning melted bismuth from a spinneret onto a rotating balloon. In addition to bismuth, other radiopaque materials may be rotational spun onto a balloon. The spinneret and other components may be configured for use in connection with metal of various melting points. For example, in some embodiments, metals with a melting point of less than about 1200 degrees C., or metals with melting points of less than about 400 degrees C., may be rotational spun in a similar manner. Furthermore, a radiopaque band or portion may comprise a ribbon or thin film of radiopaque material placed between layers of a balloon or coupled to one or more layers of a balloon. A ribbon or thin film of radiopaque material can be applied to a polymeric layer using heat, adhesive, compression, or other methods.

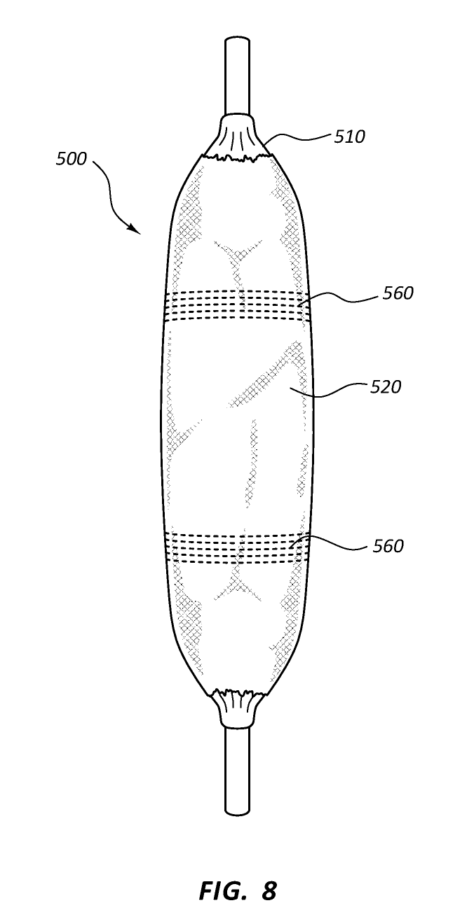

A balloon may have a radiopaque material deposited in a strip or other localized location on the balloon. For example, FIG. 8 is a front view of a balloon 500 comprising a first balloon layer 510 and a second balloon layer 520 with two radiopaque bands 560 deposited thereon. The radiopaque bands 560 may be deposited or otherwise disposed directly on any layer of a balloon, and may have additional layers of material disposed on top of the bands. For example, embodiments wherein the bands 560 are disposed on the second balloon layer 520 and embodiments wherein the bands 560 are disposed between the first balloon layer 510 and the second balloon layer 520 are within the scope of this disclosure.

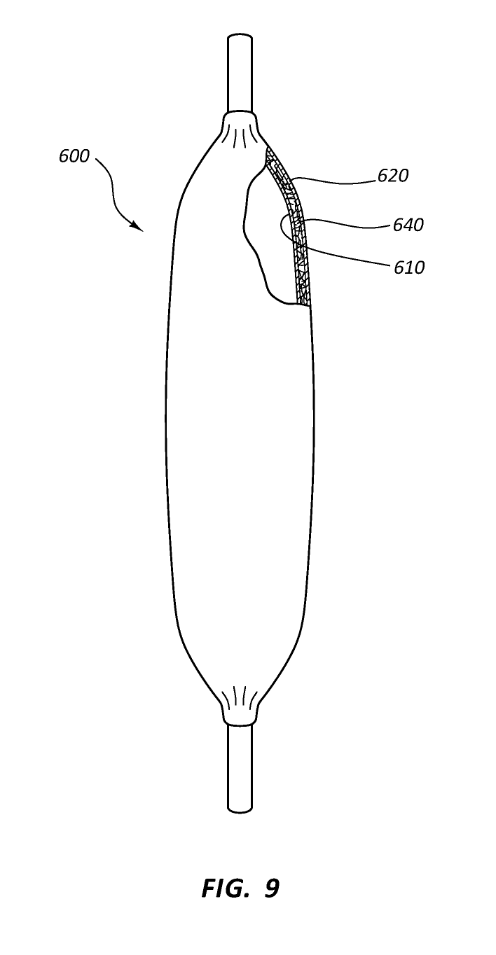

In some embodiments, a balloon may first be coated with a layer of rotational spun (or otherwise serially deposited) fibers, then the coating of rotational spun fibers covered with an additional top coat. For example, FIG. 9 is a partial cut-away view of a balloon 600 comprising a first balloon layer 610 coated with fibers 640, then covered with an outer layer 620. The outer layer 620 may comprise a dip or spray coat in some embodiments.

In still further embodiments, layering processes may be repeated to create a construct with any number of alternating fiber layers and additional layers. A layer may be added on a fiber layer to, for example, seal the fiber coat, to further bind the fibers to the balloon substrate, and/or create a smooth exterior surface. In some embodiments an outermost layer or “top coat” may be configured to be lubricious in nature, hydrophilic, or both. A top coat may be configured to facilitate delivery of the balloon through a delivery lumen, as a smooth or lubricious coating may make it easier to advance the balloon through the delivery lumen and/or facilitate the use of relatively smaller delivery lumens.

One exemplary embodiment may comprise a PEBAX balloon, first coated with rotational spun nylon fibers, then covered with a polyurethane top coat. Other exemplary top coat materials include: polyurethane, PEBAX, urethane, silicone, acrylates, Kapton, Kevlar, Elvamide, and PTFE derivates including those sold under the trade name Teflon. The top coat layer may be applied by dipping, spraying, brushing, and so forth. In some embodiments, the top coat material may be dissolved in a solvent prior to application. For example, Kevlar may be first dissolved in sulfuric acid, then applied to the fiber coat of a balloon.

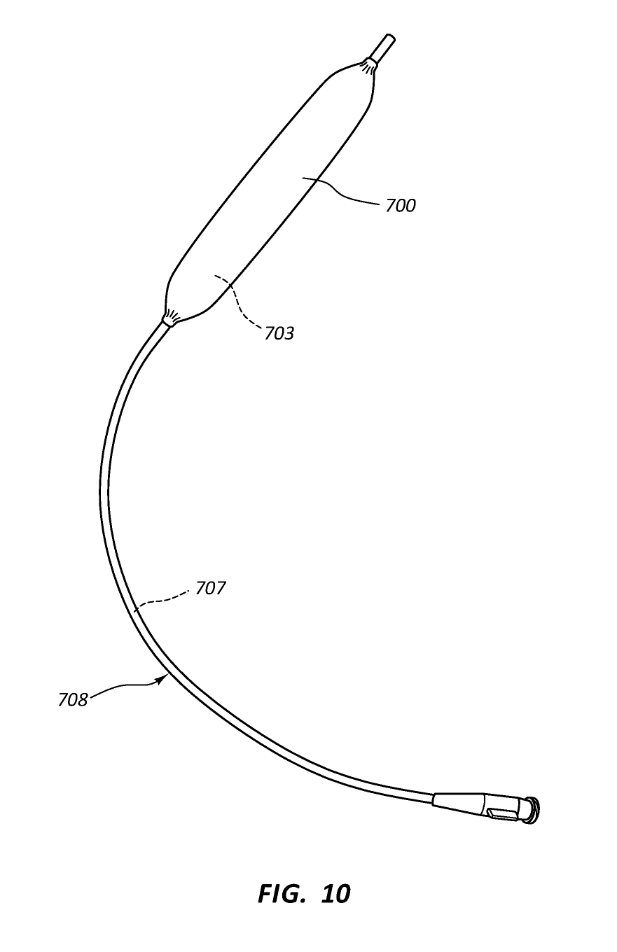

FIG. 10 is a perspective view of a balloon 700 coupled to a catheter 708. Any balloon comprising any covering, coating, or construct described herein may be coupled to a catheter to facilitate, for example, use and inflation. Any balloon or medical device disclosed herein may be used with a wide variety of other medical appliances, such as delivery catheters, inflation catheters, and so forth. In the embodiment shown in FIG. 10, the catheter 708 may be configured to inflate the balloon 700 during a therapy and/or to facilitate the advancement of the balloon 700 within a body lumen or delivery lumen. The catheter 708 may include a catheter lumen 707 that is in fluid communication with an interior portion 703 of the balloon 700, and the catheter 708 can be connected to an inflation device for delivering inflation fluid to the balloon via the catheter lumen 707.

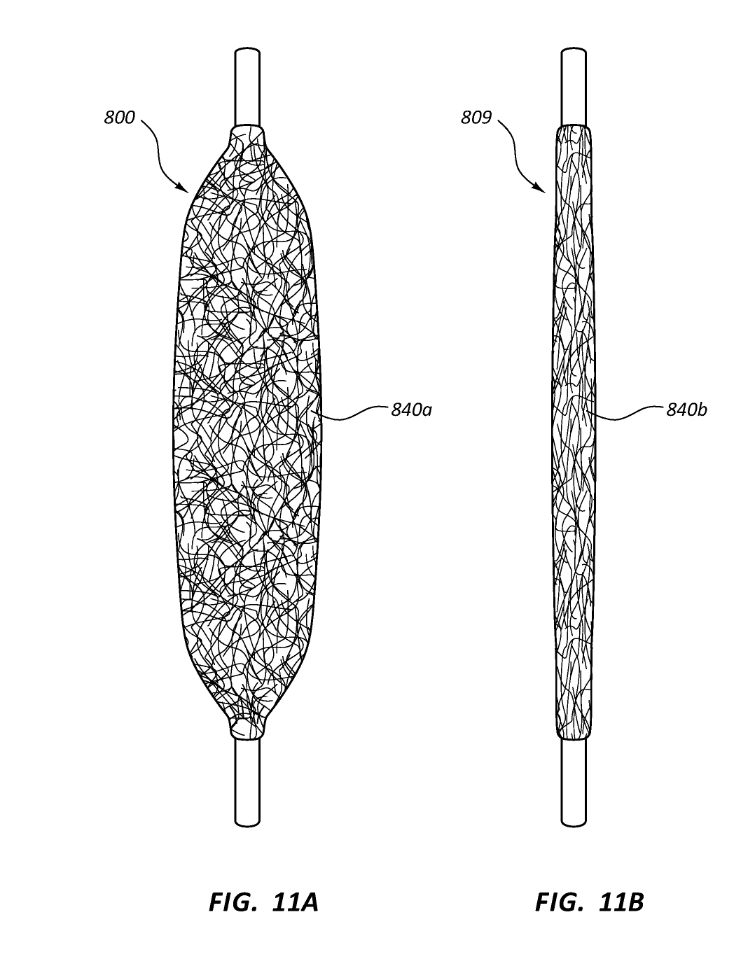

In some embodiments, fibers may also be serially deposited onto a parison, or other segment of unexpanded material, before the parison is expanded to form a balloon. For example, FIG. 11A is a front view of a fiber coated balloon 800 and FIG. 11B is a front view of a fiber coated unexpanded parison 809. In some embodiments, the parison 809 may be coated with a fiber coating 840b before the parison 809 is expanded to form a balloon, by collecting fibers on a rotating parison. The parison can be oriented horizontally, vertically, or otherwise. The fiber coated parison may then be placed in a balloon blowing mold and blown to the desired mold shape. The fibers deposited on the parison may be stretched as the balloon is blown resulting in a fibrous coating in tension around the balloon. For example, the fiber coating 840a shown on balloon 800 would comprise a layer in tension if the coating 840a were disposed on a parison which was expanded to form balloon 800. In some embodiments, the mold can be heated to partially melt the fibers of the fiber coating 840 into the wall of the balloon 800.

Any of the embodiments disclosed herein may comprise one of more layers modified by various additional processing steps, methods, procedures, and systems for serially deposited fiber mats, such as electrospun or rotational spun mats. Materials comprising serially deposited fiber mats which have been processed by any of the methods or systems described below are likewise within the scope of this disclosure. These processes and materials may be used to create multilayered constructs, including balloon constructs, having one or more layers of serially deposited fiber material which has been post processed as described below and/or having one or more layers of serially deposited fiber material which has not been post processed. The post processing methods and related materials described below describe various methods of modifying the material properties of serially deposited fiber layers to, for example, change the strength of the material, change the surface characteristics of the material, change the porosity of the material, set the material in a particular geometry or shape, and so forth. Further, though specific disclosure below may refer generally to membranes or specifically to exemplary structures such as tubes, application of any of the description below to any structure or device, including balloons, is within the scope of this disclosure.

Serially deposited fiber mats may comprise a membrane in the form of a sheet, a sphere, a strip, or any other geometry. Examples of membranes include, but are not limited to, serially deposited fiber mats or lattices forming sheets, strips, tubes, spheres, covers, layers, and so forth. Additionally, any material which can be serially deposited as fibers may be processed as described below.

Additionally, as used herein, references to heating a material “at” a particular temperature indicate that the material has been disposed within an environment which is at the target temperature. For example, placement of a material sample in an oven, the interior of the oven being set at a particular temperature, would constitute heating the material at that particular temperature. While disposed in a heated environment, the material may, but does not necessarily, reach the temperature of the environment. The term “about,” as used herein in connection with temperature, is meant to indicate a range of ±5 degrees C. around the given value. The term “about” used in connection with quantities or values indicates a range of ±5% around the value.

Serially deposited membranes may be processed to alter the strength or other characteristics of the material by stretching the membrane in one or more directions. In some embodiments the membrane may initially be sintered after it is serially deposited. The membrane may then be heated at a particular temperature prior to further processing of the membrane. As further outlined below, heating and stretching a membrane of serially deposited fibers may tend to cause increased strength in the direction the membrane is stretched. In some embodiments, the material may also exhibit increased fiber alignment in the direction of stretching.

Temperatures at which materials may be heated prior to processing may vary depending on the material and depending on the desired characteristics of the material after processing. For example, a polymeric membrane may show more or less fiber alignment after processing depending on various factors, such as the temperature at which the materials are heated. In some instances a membrane may be heated at a temperature at or above the crystalline melt point of the material comprising the membrane, though it is not necessary to heat the material as high as the crystalline melt temperature to stretch process the material.

In the case of polymeric materials which are sintered, the step of heating the membrane may be performed as a separate and distinct step from sintering the membrane, or may be done as the same step. For example, it is within the scope of this disclosure to process a membrane directly after sintering the membrane, while the membrane is at an elevated temperature due to the sintering process. It is likewise within the scope of this disclosure to obtain a previously sintered membrane which may have been previously cooled to ambient or room temperature, then heat the membrane as part of a heating and stretching process.

Membranes or any other mat or lattice of serially deposited fibers may be stretched in any direction as part of a heating and stretching process. For example, a tubular membrane may be stretched in the axial/longitudinal direction, the radial direction, or any other direction. Further, it is within the scope of this disclosure to stretch a membrane in multiple directions, either simultaneously or as part of separate steps. For example, the a tubular membrane may be stretched both axially and radially after the membrane is initially heated, or the membrane may be stretched in these or other directions as part of distinct and separate steps. Additionally, the membrane may be heated multiple times during such a process.

Various methods, modes, mechanisms, and processes may be utilized to apply forces to stretch materials. For example, force may be applied through mechanical, fluidic, electro-magnetic, gravitational, and/or other mechanism or modes. In embodiments wherein force is applied through fluidic interaction, a pressurized gas or liquid could be used to generate the force while the material is at an elevated temperature. The fluid may be stagnant or recirculating. Further, the fluid may be used to heat and/or cool the material. For example, the liquid may be used to rapidly cool the material, locking the microstructure and geometry. Additionally, stretching of fibers deposited on a parison or other deflated balloon structure may be stretched by inflating the underlying balloon, including inflating the balloon while the materials are at an elevated temperature.

A heated and stretched membrane may be held in a stretched position while the membrane cools. For example, a membrane may be heated at an elevated temperature prior to stretching, stretched while the membrane is at an elevated temperature, then held in the stretched position while the membrane cools to an ambient temperature, such as room temperature. Depending on the process, when the membrane is stretched, it may be at a temperature lower than the temperature at which it was heated, and it may or may not cool completely to the ambient temperature while the position is held.

Processing a mat or lattice of serially deposited fibers as by heating and stretching may alter various material properties of the mat or lattice. For example, and as further outlined below, heating and stretching a fiber mat may increase the durability of the material, increase the smoothness of the material, increase handling characteristics, increase the tensile strength of the material, increase resistance to creep, or otherwise alter the material. Further, in some embodiments, heating and stretching the material tends to align a portion of the fibers which comprise the mat in the direction the material is stretched. This alignment of the microstructure and/or nanostructure of the material may impact microscale and/or nanoscale interactions between the mat and other structures, such as body cells. Fiber alignment may likewise alter the flow characteristics of a fluid flowing in contact with the mat. For example, a tubular membrane configured to accommodate blood flow may exhibit different flow conditions through the tube if the fibers are aligned by heating and stretching as compared to randomly disposed fibers.

Additionally, heating and stretching a mat may or may not tend to align the fibers in the direction the material is stretched. In some embodiments, the degree of fiber alignment may be related to the temperature at which the mat was heated prior to stretching. Still further, stretching a mat in multiple directions may tend to maintain random fiber disposition of a mat in embodiments wherein the original mat exhibited generally random fiber disposition.

Regardless of whether heating and stretching tend to align the fibers in the direction the mat was stretched, the mat may exhibit different properties in a stretched direction as compared to a non-stretched direction. For example, the mat may exhibit increased tensile strength and/or increased resistance to creep in the stretched direction while these properties may be generally unchanged or decreased in a non-stretched direction. Further, stretching may increase the porosity of a mat of serially deposited fibers. In some embodiments, stretching may increase the porosity of a mat by up to 10 times the original porosity, including up to eight times, up to six times, up to four times, and up to two times the original porosity. In some embodiments, a mat may be stretched while at room temperature to increase porosity, to increase strength, or to modify other properties of the mat.

Additionally, in some embodiments, a tubular membrane heated and stretched in the axial direction may exhibit greater tensile strength in the axial direction as compared to the properties of the membrane prior to heating and stretching. In this example, the tensile strength in the radial direction, however, may be similar to the tensile strength of the membrane in that direction prior to heating and stretching. Thus, the membrane may have similar properties in both these directions prior to heating and stretching, but may exhibit greater tensile strength in the axial direction after heating and stretching. In some embodiments, the tensile strength of the membrane is 150% to 300% that of the membrane prior to heating and stretching in the direction of stretching. For example, the tensile strength of the membrane is at least 150%, at least 200%, at least 250% or at least 300% that of the membrane prior to heating and stretching in the direction of stretching. In some embodiments, a mat may exhibit decreased tensile strength or other changes in properties in a non-stretched direction disposed perpendicular to the direction of stretching, as compared to those properties prior to stretching.

In some embodiments, a material is stretched in multiple directions to increase strength or otherwise alter the properties in those directions. In other embodiments, heating and stretching change the properties in only one direction. For example, a tube may be configured to be bolstered against creep in the radial direction, without substantially affecting the material properties in the axial direction. Again, in some instances an increase in particular properties in a first direction is correlated with a decrease in one or more of the same properties in a second direction.

Additionally, materials having different properties in different directions may be combined to create a composite construct. For example, a composite construct comprising at least one layer of axially stretched material and at least one layer of radially stretched material may exhibit increased strength in both directions. Various layers having various properties may be combined to tailor the properties of the resultant construct. It is within the scope of this disclosure to bond adjacent layers through various processes, including use of tie layers disposed between layers and bonded to each layer, heating adjacent layers to create fiber entanglement, use of adhesives, and so forth. Fluorinated ethylene propylene (FEP) may be used as a tie layer in some embodiments. Further, expanded PTFE may be used as a tie layer in some embodiments. One embodiment of a composite tube can be created by helically or cigar wrapping a tube of serially deposited fibers (un-stretched) with a film of heat and stretch processed material, creating a porous luminal layer and a strong creep resistant reinforcement layer. Additionally, layers (such as an impervious layer and/or a porous abluminal layer) may be added to the construct as well. Each layer may be configured to optimize a physiologic interaction, for example.

Multilayered constructs may further comprise reinforcing structures, such as metal scaffolds or frames. In some embodiments, a reinforcing structure may comprise one of: Nitinol, stainless steel, or titanium. Any layer of a construct may be configured to be a blood contacting layer. Blood contacting layers may be configured to interact with the blood or other biological elements and may be configured with certain flow characteristics at the blood interface. Further, any layer of a multilayered construct may be configured to be impermeable to tissue or fluid migration. For example an impermeable tie layer may be disposed between porous inner and outer layers of a construct.

Single layer devices or multilayered constructs within the scope of this disclosure may comprise tubes, grafts, stents, stent grafts, vascular grafts, patches, prosthetics, or any other medical appliance. Medical appliances configured for oral surgery and/or plastic surgery are also within the scope of this disclosure.

Again, heat and stretch processing may increase strength in the stretched direction while decreasing strength in a direction perpendicular to the stretched direction. For example, a tubular membrane stretched in the axial direction may exhibit greater strength in the axial as opposed to the radial direction. Further, a membrane so processed may exhibit greater elasticity or “spring” in the non-stretched direction oriented perpendicular to the stretched direction.

Heating and stretching a mat or lattice of serially deposited fibers may tend to decrease the thickness of the mat or lattice. For example, a tubular mat stretched in the range from 200% to 450% may exhibit a decrease in material thickness of between 10% and 90%, including from 20% to 80% and from 40% to 60%. Embodiments within these ranges may not exhibit holes or defects from the stretching process, and the overall surface quality of the material may be maintained after stretching. Further, these ranges are intended to correlate the degree of stretching and the decrease in material thickness, not to constitute upper or lower bounds. Materials may be stretched further than the given range to further decrease the material thickness, for instance.

As stated above, it is within the scope of this disclosure to heat and stretch various serially deposited fiber mats comprising various materials. Many of the examples discussed below refer particularly to PTFE fiber mats which have been processed in a variety of ways. These examples, or any other example referencing PTFE, may analogously apply to other materials as well. Specific temperatures for heating or otherwise processing a material may be analogously applied to other materials by considering the material properties (such as melting point) of such materials and analogizing to the examples below.

Generally, serially deposited PTFE fiber mats may be heated at temperatures between about 65 degrees C. and about 400 degrees C. while heating and stretching the mats. For example, serially deposited PTFE fiber mats may be heated at temperatures above about 65 degrees C., above about 100 degrees C., above about 150 degrees C., above about 200 degrees C., above about 250 degrees C., above about 300 degrees C., above about 350 degrees C., above about 370 degrees C., and above about 385 degrees C. Additionally, serially deposited PTFE fiber mats may be stretched at room temperature (22 degrees C.) without heating.

Serially deposited PTFE mats may be stretched from 150% to 500% of the initial length of the mat in the direction of stretching, including stretching mats to between 200% and 350%, between 250% and 300%, and between 300% and 500% of the original length of the mats in the direction of stretching. The amount of length change may be related to the temperature at which the mat is heated, the force applied when the mat is stretched, the original thickness of the mat, and the rate at which the mat is stretched.

Processing serially deposited fiber mats or lattices through heating and stretching may impact various properties of the mats. Tensile strength, resistance to creep, elasticity, and so forth may all be impacted. In some embodiments, processed mats are used as layers of multilayered constructs to provide particular properties in a particular direction.

The temperature at which mats of serially deposited PTFE fibers are heated may affect the tendency of the fibers of the mats to align after the mats are stretched. Higher temperatures generally correlate with increased fiber alignment. Generally, PTFE mats heated at or above 370 degrees C. exhibit more fiber alignment than mats heated at temperatures lower than 370 degrees C. Additionally, an increase in tensile strength is correlated with heating and stretching PTFE, whether or not the mat was heated at 370 degrees C. or more. The amount of the increase in tensile strength may be affected by the temperature at which the mat was heated and the amount the material was stretched.

Serially deposited fibers may be set in various geometries by constraining the fibers in a particular geometry and heating the fibers. For example, in some embodiments, constraining a previously sintered (or otherwise structurally set) mat or lattice of serially deposited fibers in a particular configuration, softening the material of the mat or lattice (for example by heating), and allowing the material to reset may result in a “memory” effect wherein the material retains at least a portion of the constrained geometry. Materials may be shape-set as described herein whether or not the materials have been heated and stretched as described above.

In embodiments comprising serially deposited polymeric fibers, heating the material at about the crystalline melt point of the material may facilitate setting of the geometry.

In one exemplary embodiment, a tubular membrane may be serially deposited on a mandrel, sintered, and removed from the mandrel. Though this specific example includes a tubular membrane, the present disclosure also applies to sheets, spheres, and other geometries of serially deposited fiber mats. The tubular membrane of sintered serially disposed polymeric fibers may then be constrained in a variety of configurations. For example, the membrane may be compressed onto a mandrel such that the tubular membrane is compressed along a shorter length, tending to create annular ridges or corrugations along the length of the membrane.

Once the membrane is constrained into the desired shape, the membrane may be heated while constrained. After heating and cooling, the membrane may tend to retain the constrained shape. A tubular membrane set in a corrugated shape may exhibit elasticity between the ends of the membrane due to the corrugation. When pulled in the axial direction (opposite the direction the membrane was compressed prior to heat-setting) then released, the membrane will tend to return to the heat-set corrugated configuration.

Furthermore, in the case of a corrugated tubular membrane, corrugations may facilitate bending of the membrane. Specifically, the annular corrugations may both reinforce the membrane and provide elasticity such that the membrane can bend in a variety of configurations without kinking. A corrugated membrane may be disposed over a balloon in some embodiments. Further, a balloon material may be heated and set in a corrugated or other geometry.

### EXAMPLES

The specific examples included below are for illustrative and explanatory purposes, and are not to be considered as limiting to this disclosure.

### Example 1

A first balloon having a wall comprising a single layer of PEBAX was inflated until the balloon ruptured. The maximum inflation pressure was recorded as the burst strength of the first balloon. The burst strength of the first balloon was measured to be about 12 ATM.

A second balloon having a wall comprising two adjacent layers of PEBAX was then tested. The second balloon was formed by inflating a primary balloon, then inserted a secondary balloon into the primary balloon. The secondary balloon was deflated and folded to a low-profile configuration to facilitate insertion into the primary balloon. The secondary balloon was then inflated within the primary balloon, forming a single balloon (the second balloon) having a wall comprising two adjacent layers. The primary balloon and the secondary balloon were substantially similar in size and wall thickness; thus the second balloon's total wall thickness was double that of the first balloon.

The second balloon was similarly inflated until the balloon ruptured, and the maximum inflation pressure recorded. The burst strength of the second balloon was measured to be about 20 ATM, an increase of about 8 ATM over the first, single walled balloon.

### Example 2

A balloon having a wall comprising three adjacent layers of PEBAX was formed by first inflating a primary balloon, then inserting a secondary balloon into the primary balloon. The secondary balloon was deflated and folded to a low-profile configuration to facilitate insertion into the primary balloon. The secondary balloon was then inflated within the primary balloon, similar to the second balloon of Example 1. A tertiary balloon was then deflated and inserted into the secondary balloon. The tertiary balloon was then inflated within the secondary balloon, forming a single balloon having a wall comprising three adjacent layers. The primary, secondary, and tertiary balloons were each substantially similar in size and wall thickness to the first balloon of Example 1; thus the three layer balloon's total wall thickness was triple that of the first balloon of Example 1.

The three layer balloon was then inflated until the balloon ruptured and the maximum inflation pressure measured. The burst strength of the three layer balloon was measured to be about 29 ATM, an increase of about 17 ATM over the single walled first balloon of Example 1.

### Example 3

A single layer PEBAX balloon was obtained and inserted into a tube of stainless steel mesh. The balloon was then inflated until the one or both components ruptured. The stainless steel mesh tube ruptured at 14 ATM, however, the PEBAX balloon did not. This result was compared to the first single layer PEBAX balloon of Example 1, which ruptured at 12 ATM.

It appeared that, because the PEBEX is much more elastic than the stainless steel, the stainless steel was essentially bearing the pressure load during inflation of the PEBAX balloon. The burst strength of the less elastic material thus appeared to control, as the less elastic material tends to bear substantially the entire pressure load during testing.

### Example 4

Polyamide 6 (Nylon 6) was dissolved in 1,1,1,3,3,3-hexafluoro-2-propanol to create three solutions, one comprising 9% nylon 6 by weight, one comprising 13% nylon 6 by weight, and one comprising 15% nylon 6 by weight. Each solution was then rotational spun from a spinneret rotating between about 7500 RPM and about 8000 RPM. The spinneret was configured with 26 gauge needle orifices.

Generally, it was observed that the higher weight percent nylon 6 solutions spun better at higher rotational speeds as the solution was more viscous. Each solution produced relatively well defined fibers. It was further observed that the higher concentration solutions produced stronger fibers. Finally, it was observed that, while rotational spinning the solution, the solvent tended to evaporate relatively quickly.

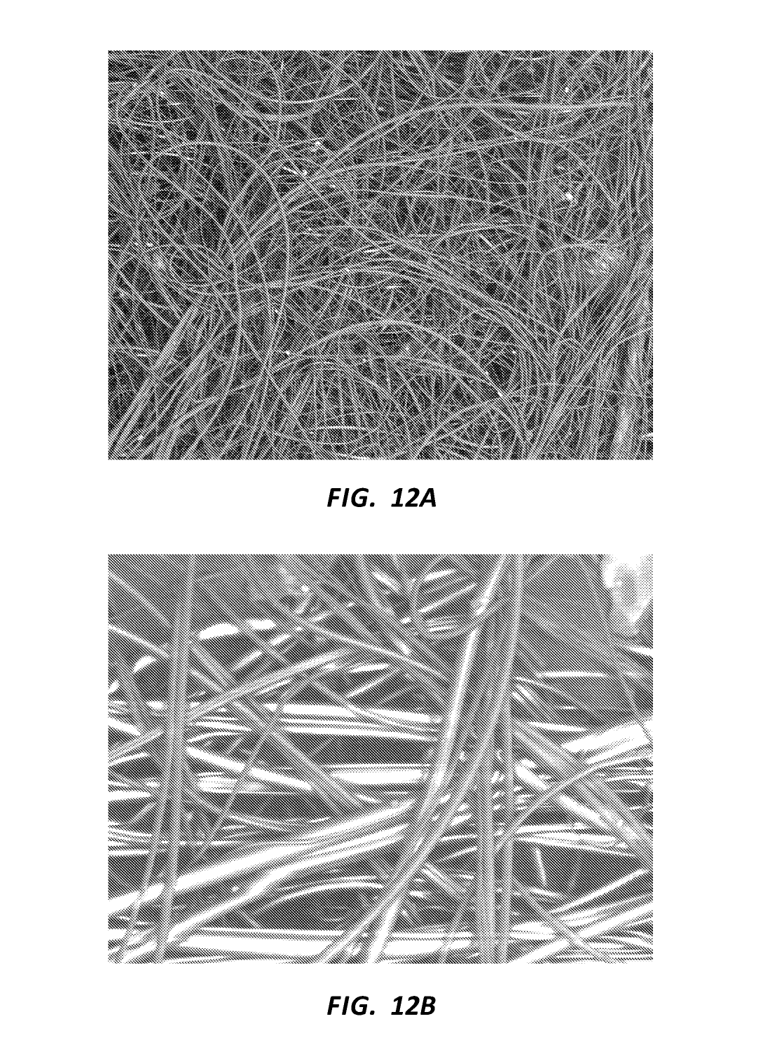

The second solution, comprising 13% nylon 6 by weight, was rotational spun at 7500 RPM onto a horizontally mounted balloon rotating at about 200 RPM. FIG. 12A is a scanning electron micrograph (SEM) of a nylon coating at 170× magnification. FIG. 12B is an SEM of the nylon coating at 950× magnification.

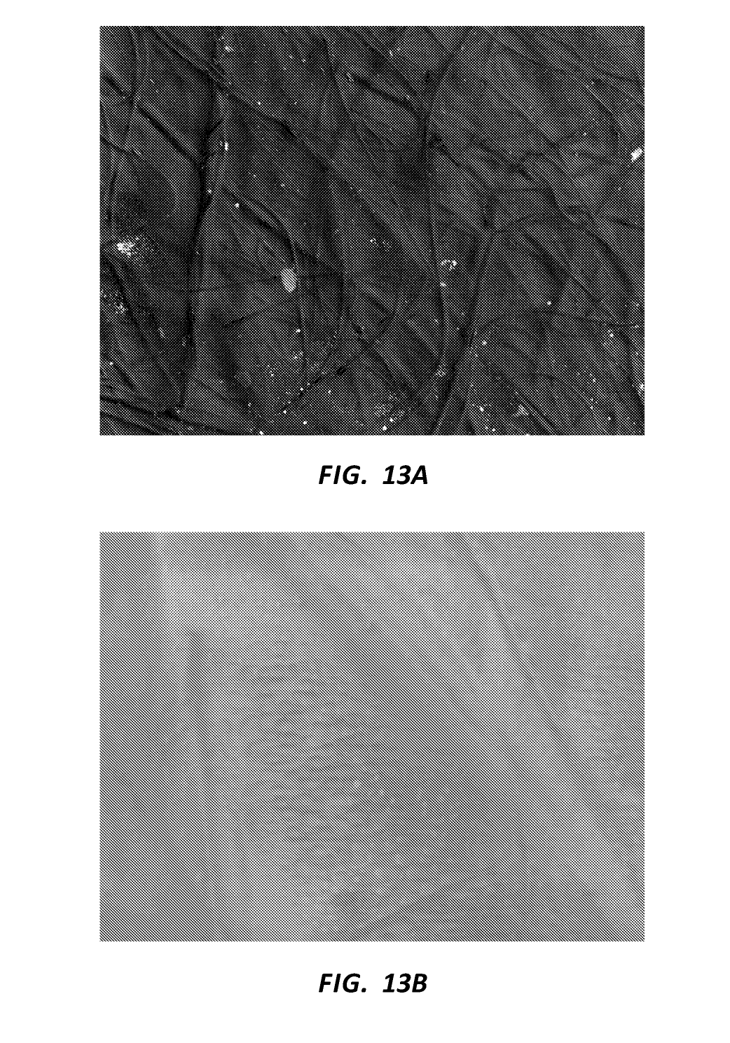

A nylon covering similar to that described in connection with FIGS. 12A and 12B was further covered with a urethane top coat. FIG. 13A is an SEM of this rotational spun nylon coating covered with a urethane top coat at 170× magnification. FIG. 13B is an SEM of the nylon coating and urethane top coat at 950× magnification.

### Example 5

Nylon 6 was dissolved in formic acid (>88.0%) to create three solutions, one comprising 21% nylon 6 by weight, one comprising 15% nylon 6 by weight, and one comprising 10% nylon 6 by weight. Each solution was then rotational spun from a spinneret rotating between about 7500 RPM and about 8000 RPM. The spinneret was configured with 26 gauge needle orifices.

It was observed that each of these solutions produced fibers. The fibers were each observed to have relatively small diameters. Finally, it was observed that lower concentrations of nylon 6 did not produce as well defined fibers as higher concentrations.

### Example 6

A matrix of PTFE, PEO, and bismuth subcarbonate was rotational spun according to the following procedure. A solution of bismuth subcarbonate in water, comprising 60% bismuth subcarbonate by weight, was created by adding 12 grams of bismuth subcarbonate to 20 mL of water. 3.5 grams of PEO and 4.29 mL of de-ionized water were added to 20 mL of the bismuth subcarbonate-water solution to create a 0.144 g/mL PEO/bismuth subcarbonate mixture. 20 mL of 60 weight percent PTFE dispersion was then added to the mixture and the resultant solution was rotational spun. The solution was spun at between about 2000 RPM and about 3000 RPM for about one minute onto an aluminum foil collector. The resultant matrix demonstrated an exemplary procedure for including a radiopaque agent, such as bismuth, into a fiber matrix.

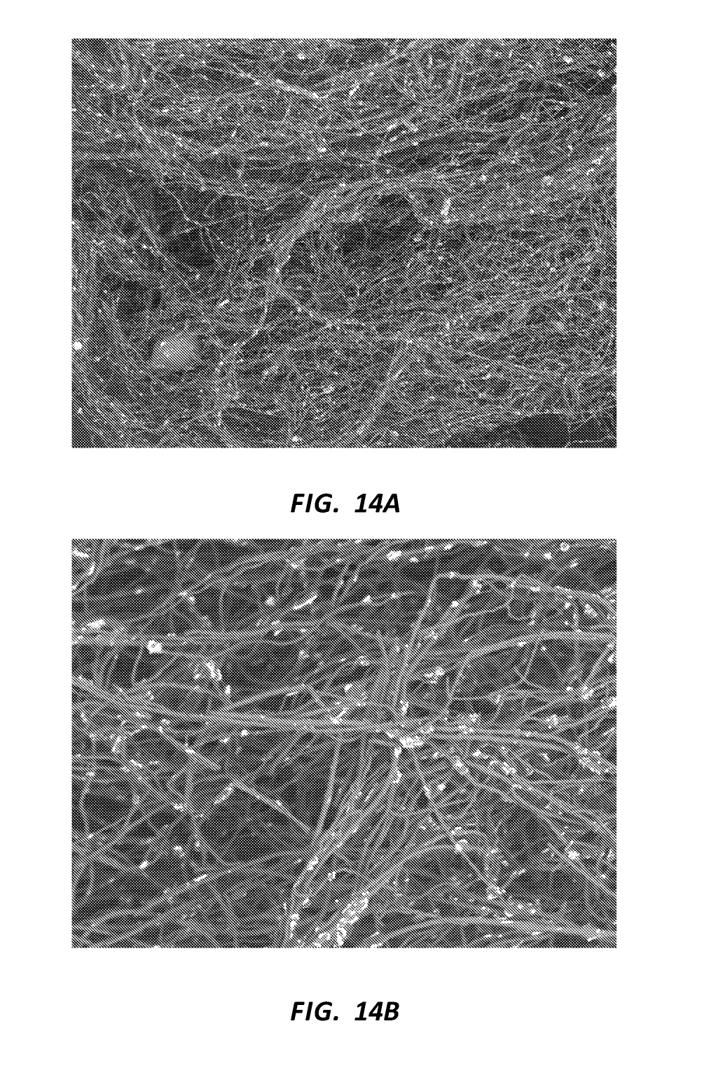

FIG. 14A is an SEM of the rotational spun matrix of PTFE, PEO, and bismuth subcarbonate at 170× magnification. FIG. 14B is an SEM of the matrix at 950× magnification. Both SEMs show the particles of bismuth subcarbonate.

### Exemplary Embodiments

The following embodiments are illustrative and exemplary and not meant as a limitation of the scope of the present disclosure in any way.

I. Balloon Catheter

In one embodiment, a balloon catheter comprises an inflatable balloon portion including a wall portion comprising a first layer and a second layer, the second layer being formed separately from the first layer; and a catheter portion comprising a lumen in fluid communication with the inflatable balloon portion and configured to deliver inflation fluid from an inflation device to the inflatable balloon portion.

The first layer and the second layer may be unconstrained relative to each other over part of the wall portion, such that the first layer can slide with respect to the second layer.

The first layer and the second layer may be formed of a thermoplastic polymer material.

The first layer and the second layer may also be formed of the same material.

The second layer may alternatively be formed of a different material than the first layer, such that a modulus of elasticity of the second layer is no more than 20%, no more than 10%, or no more than 5% different than a modulus of elasticity of the first layer.

The wall portion may further comprise a third layer.

Additionally, the wall portion may further comprise a fourth layer.

The first layer may comprise a first balloon and the second layer may comprise a second balloon disposed within the first balloon.

The balloon catheter may further comprise a fiber layer disposed on an outer surface of the first layer.

The fiber layer may comprise rotational spun nano-fibers or micro-fibers.

The rotational spun nano-fibers or micro-fibers may comprise a material selected from at least one of the following: polyamide, aromatic polyimide, polyethylene and polypropylene.

The rotational spun nano-fibers or micro-fibers may comprise a polyamide.

The polyamide may be nylon 6 or nylon 6-6.

The balloon catheter may further comprise a polyurethane coat or an Elvamide coat over the fiber layer.

The balloon catheter may further comprise a Kapton layer disposed on an outer surface of the first layer.

The balloon catheter may further comprise a rotational spun radiopaque material disposed on the inflatable balloon portion.

The radiopaque material may comprise a bismuth ring.

The radiopaque material may comprise a polymer fiber coated with bismuth subcarbonate.

In another embodiment, a balloon catheter comprises an inflatable balloon portion including a wall portion comprising a first thermoplastic polymer base layer and a second fiber layer disposed on an outer surface of the first layer; and a catheter portion comprising a lumen in fluid communication with the inflatable balloon portion and configured to deliver inflation fluid from an inflation device to the inflatable balloon portion.

The fiber layer may comprise rotational spun nano-fibers or micro-fibers.

The rotational spun nano-fibers or micro-fibers may comprise a material selected from at least one of the following: polyamide, aromatic polyimide, polyethylene and polypropylene.

In one embodiment, the rotational spun nano-fibers or micro-fibers comprise a polyamide.

The polyamide may be nylon 6 or nylon 6-6.

The balloon catheter may further comprise a polyurethane coat over the fiber layer.

II. Methods of Manufacture

In one embodiment, a method for manufacturing a balloon catheter comprises forming a first thermoplastic polymer balloon layer; forming a second thermoplastic polymer balloon layer that is disposed inside the first thermoplastic polymer balloon layer, such that the first and second thermoplastic polymer balloon layers comprise an inflatable balloon portion; and coupling a catheter comprising a lumen to the inflatable balloon portion such that the lumen is in fluid communication with the inflatable balloon portion.

The step of forming the second thermoplastic polymer balloon layer may comprise inflating the first thermoplastic polymer balloon layer; forming the second thermoplastic polymer balloon layer separate from the first thermoplastic polymer balloon layer; collapsing the second thermoplastic polymer balloon layer; inserting the second thermoplastic polymer balloon layer into the first thermoplastic polymer balloon layer; and inflating the second thermoplastic polymer balloon layer.

The step of forming the second thermoplastic polymer balloon layer may comprise forming the second thermoplastic polymer balloon layer within the first thermoplastic polymer balloon layer.

The second thermoplastic polymer balloon layer may be formed by expanding a heated thermoplastic tube.

At least a portion of the method for manufacturing the balloon catheter may be completed in a vacuum chamber.

The method may further comprise applying negative gauge pressure to remove air from the inflatable balloon portion.

The method may further comprise rotational spinning a nano-fiber or micro-fiber layer onto the first thermoplastic polymer balloon layer.

The rotational spun nano-fibers or micro-fibers may comprise a material selected from at least one of the following: polyamide, aromatic polyimide, polyethylene and polypropylene.

In one embodiment, the rotational spun nano-fibers or micro-fibers comprise a polyamide.

The method may further comprise dissolving the polyamide into an organic or inorganic solvent to create a solution or dispersion.

The solvent may comprise hexafluoro propanol.

The solution with hexafluoro propanol may comprise 5% to 30% polyamide by weight.

The solution with hexafluoro propanol may comprise 10% to 20% polyamide by weight.

The solvent may comprise formic acid.

The solution with formic acid may comprise 10% to 30% polyamide by weight.

The solution with formic acid may comprise 15% to 25% polyamide by weight.

The solution with formic acid may comprise 20% to 25% polyamide by weight.

The method may further comprise rotational spinning bismuth(s) or a bismuth polymer dispersion onto the first thermoplastic polymer balloon layer.

The method may further comprise rotating the first thermoplastic polymer balloon layer.

The first thermoplastic polymer balloon layer may be rotated about an axis parallel to an axis of rotation of a rotational spinning apparatus.

The first thermoplastic polymer balloon layer may be rotated about an axis orthogonal to an axis of rotation of a rotational spinning apparatus.

According to another embodiment, a method for manufacturing a balloon catheter may comprise forming a first thermoplastic polymer balloon layer; rotational spinning a nano-fiber or micro-fiber layer onto the first thermoplastic polymer balloon layer; and coupling a catheter comprising a lumen to the inflatable balloon portion such that the lumen is in fluid communication with the inflatable balloon portion.

The rotational spun nano-fibers or micro-fibers may comprise a material selected from at least one of the following: polyamide, aromatic polyimide, polyethylene and polypropylene.

In one embodiment, the rotational spun nano-fibers or micro-fibers comprise a polyamide.

The method may further comprise dissolving the polyamide into an organic or inorganic solvent to create a solution.

The solvent may comprise hexafluoro propanol.

The solution with hexafluoro propanol may comprise 5% to 30% polyamide by weight.

The solution with hexafluoro propanol may comprise 10% to 20% polyamide by weight.

The solvent may comprise formic acid.

The solution with formic acid may comprise 10% to 30% polyamide by weight.

The solution with formic acid may comprise 15% to 25% polyamide by weight.

The solution with formic acid may comprise 20% to 25% polyamide by weight.

The method may further comprise rotational spinning bismuth(s) or a bismuth polymer dispersion onto the first thermoplastic polymer balloon layer.

The method may further comprise rotating the first thermoplastic polymer balloon layer.

The first thermoplastic polymer balloon layer may be rotated about an axis parallel to an axis of rotation of a rotational spinning apparatus.

The first thermoplastic polymer balloon layer may be rotated about an axis orthogonal to an axis of rotation of a rotational spinning apparatus.

Without further elaboration, it is believed that one skilled in the art can use the preceding description to utilize the present disclosure to its fullest extent. The examples and embodiments disclosed herein are to be construed as merely illustrative and exemplary and not as a limitation of the scope of the present disclosure in any way. It will be apparent to those having skill in the art, and having the benefit of this disclosure, that changes may be made to the details of the above-described embodiments without departing from the underlying principles of the disclosure herein.

1. A balloon catheter, comprising:
an inflatable balloon portion including a wall portion comprising a first layer and a second layer, the second layer being formed separately from the first layer; and
a catheter portion comprising a lumen in fluid communication with the inflatable balloon portion and configured to deliver inflation fluid from an inflation device to the inflatable balloon portion.

2. The balloon catheter of claim 1, wherein the first layer and the second layer are unconstrained relative to each other over part of the wall portion, such that the first layer can slide with respect to the second layer.

3. The balloon catheter of claim 1, wherein the first layer and the second layer are formed of a thermoplastic polymer material.

4. The balloon catheter of claim 3, wherein the first layer and the second layer are formed of the same material.

5. The balloon catheter of claim 3, wherein the second layer is formed of a different material than the first layer, such that a modulus of elasticity of the second layer is no more than 20% different than a modulus of elasticity of the first layer.

6. The balloon catheter of claim 1, wherein the first layer comprises a first balloon and the second layer comprises a second balloon disposed within the first balloon.

7. The balloon catheter of claim 1, further comprising a fiber layer disposed on an outer surface of the first layer.

8. The balloon catheter of claim 7, wherein the fiber layer comprises serially deposited nano-fibers or micro-fibers.

9. The balloon catheter of claim 8, wherein the serially deposited fibers comprise rotational spun fibers that comprise a material selected from at least one of the following: polyamide, aromatic polyimide, polyethylene and polypropylene.

10. The balloon catheter of claim 9, wherein the rotational spun nano-fibers or micro-fibers comprise a polyamide.

11. The balloon catheter of claim 10, wherein the polyamide is nylon 6 or nylon 6-6.

12. The balloon catheter of claim 7, further comprising a polyurethane coat or an Elvamide coat over the fiber layer.

13. The balloon catheter of claim 1, further comprising a Kapton layer disposed on an outer surface of the first layer.

14. The balloon catheter of claim 1, further comprising a radiopaque material disposed on the inflatable balloon portion.

15. The balloon catheter of claim 14, wherein the radiopaque material is selected from at least one of: a rotational spun bismuth ring, a ribbon, or a thin film.

16. The balloon catheter of claim 14, wherein the radiopaque material comprises a polymer fiber coated bismuth subcarbonate.

17. The balloon catheter of claim 8, wherein the serially deposited fibers comprise fibers that have been stretched in a first direction.

18. A method for manufacturing a balloon catheter, comprising:
forming a first thermoplastic polymer balloon layer;
forming a second thermoplastic polymer balloon layer that is disposed inside the first thermoplastic polymer balloon layer, such that the first and second thermoplastic polymer balloon layers comprise an inflatable balloon portion; and
coupling a catheter comprising a lumen to the inflatable balloon portion such that the lumen is in fluid communication with the inflatable balloon portion.

19. The method of claim 18, wherein forming the second thermoplastic polymer balloon layer comprises:
inflating the first thermoplastic polymer balloon layer;
forming the second thermoplastic polymer balloon layer separate from the first thermoplastic polymer balloon layer;
collapsing the second thermoplastic polymer balloon layer;
inserting the second thermoplastic polymer balloon layer into the first thermoplastic polymer balloon layer; and
inflating the second thermoplastic polymer balloon layer.

20. The method of claim 18, wherein forming the second thermoplastic polymer balloon layer comprises forming the second thermoplastic polymer balloon layer within the first thermoplastic polymer balloon layer.

21. The method of claim 18, further comprising applying negative gauge pressure to remove air from the inflatable balloon portion.

22. A method for manufacturing a balloon catheter, comprising:
forming a first thermoplastic polymer balloon layer;
serially depositing a nano-fiber or micro-fiber layer onto the first thermoplastic polymer balloon layer; and
coupling a catheter comprising a lumen to the inflatable balloon portion such that the lumen is in fluid communication with the inflatable balloon portion.

23. The method of claim 22, further comprising forming a second thermoplastic polymer balloon layer inside the first thermoplastic polymer balloon layer.

24. The method of claim 22, wherein serially depositing a nano-fiber or micro-fiber layer comprises rotational spinning polymeric fibers.

25. The method of claim 24, wherein the rotational spun nano-fibers or micro-fibers comprise a material selected from at least one of the following: polyamide, aromatic polyimide, polyethylene and polypropylene.

26. The method of claim 25, wherein the rotational spun nano-fibers or micro-fibers comprise a polyamide.

27. The method of claim 26, further comprising dissolving the polyamide into an organic or inorganic solvent to create a solution.

28. The method of claim 27, wherein the solvent is selected from hexafluoro propanol or formic acid.

29. The method of claim 28, wherein the solution comprises 5% to 30% polyamide by weight.

30. The method of claim 24, wherein the first thermoplastic polymer balloon layer is rotated about an axis orthogonal to an axis of rotation of a rotational spinning apparatus.
